(19) United States
(12) Patent Application Publication
(10) Pub. No.: US 2021/0267836 A1
(43) Pub. Date: Sep. 2, 2021

(54) Neck Bed Cervical Traction Pillow - Chiropractic Orthotic Massage Device for Neck and Shoulder Pain Trigger Point Therapy and Posture Corrector with Acupressure and Occipital Release Ridge

(71) Applicant: Oscar Raymond, Hialeah, FL (US)

(72) Inventor: Oscar Raymond, Hialeah, FL (US)

(21) Appl. No.: 16/805,936

(22) Filed: Mar. 2, 2020

(51) Int. Cl. A61H 1/02; A47G 9/10

(52) U.S. Cl. CPC A61H 1/0296; A47G 9/1081; A47G 2009/1018; A61H 2201/0134; A61H 2201/0192

(57) ABSTRACT

The present invention discloses an enhanced design of Occipital Release Tool with symmetrical estimations in stature and side width to provide enhanced stability. The assembly has occipital nodes, high intensity foam and two totally separate connectable levels which fit as blocks and enable the user to adjust each level as per individual requirements.

### COPYRIGHT NOTICE

A portion of the disclosure of this patent document contains material which is subject to copyright protection. The copyright owner has no objection to the facsimile reproduction by anyone of the patent disclosure, as it appears in the Patent and Trademark Office patent files or records, but otherwise reserves all copyright rights whatsoever.

### BACKGROUND

### Field of the Invention

The invention relates to orthotic devices. In a particular form, the present invention relates to occipital Release Tool with enlarged occipital nodes and increased firmness, is great for a variety of relaxation techniques, exercises and stretches that assist to restore and recover problematic soft tissues in the neck.

### Description of the Related Art

Existing pillow, is generally divided into daily pillow and therapy pillow, daily pillow comparatively soft comfortable but without physiotheraping effect, and common therapy pillow, often by when lying low, being obtained by the cervical vertebra of padded people and keeping or the effect of rectification cervical curvature.

At present, with the traditional pillow on the market more than a “bread” type structure, also a small amount of high head low profile neck one-piece structure and the structure of the auxiliary cylinder treatment of cervical disease. The common problem is that in the Earth's gravitational field, head and neck while sleeping difficult balancing force, the force that is not the point in the brain after head and neck. Stress points after the first bend on the brain could easily lead to cervical spine, detrimental to the prevention and control of cervical disease; stress points in the neck could easily lead to lack of sleep blood supply to the brain. Neck pillow with head high despite the low-profile integrated structure is designed taking into account the different height requirements of the neck and head, but can not vary, the neck of the head is difficult to completely balanced force. In addition side when high due to shoulder greater than the pillow can cause shoulder squeezed, affecting blood circulation.

Neck pain is very common and most of us have it at some point in our lives. Usually, neck pain is the result of holding neck in the same position for too long. At present owing to bending over one's desk working for a long time and the increase of bad activity of bowing (popular as cell phone network), make the patient of neck-shoulder pain and prolapse of cervical intervertebral disc. Numerous patients have a strong impact on its daily life and work and Spine Clinic consultation rate is increasing day by day.

There are multiple solutions that have been presented toward managing this situation. For instance, Neck Orthotic, Orthotic pillows, massage tools and other medical assemblies. Similarly, in prior art we can also see multiple inventions with their own set of advancement.

A Chinese patent 1,056,16051A on multifunctional prevention and treatment instrument for pain of neck, shoulders, waist and back is issued to Chinese inventor. The invention provides a multifunctional prevention and treatment instrument for pain of the neck, the shoulders, the waist and the back. The multifunctional prevention and treatment instrument comprises a base, a supporting part, bracket parts, a press part and a pushing and pulling part, wherein an adjusting support rod is fixedly mounted on the base; the supporting part is hinged to the adjusting support rod and comprises an upper body supporting part and a lower limb supporting part which can rotate relatively; a pair of bracket parts are symmetrically erected on two sides of the supporting part, bottom ends are hinged with the base, and a connecting rod is connected between top ends; the press part is fixed on the connecting rod and comprises a vertical guide frame fixedly connected with the connecting rod, the vertical guide frame is sleeved with a press rod sliding in the vertical direction, and load weights are mounted on the press rod; the pushing and pulling part comprises a pair of symmetrical pushing and pulling rods, the pushing and pulling rods are located at one end where the upper body supporting part is located, bottom ends of the pushing and pulling rods are hinged with the base, and the bracket parts are contacted with the pushing and pulling rods when inclined towards the end where the upper body supporting part is located. According to the prevention and treatment instrument, structures of all parts are matched for use and are simple and compact, and exercise and healthcare treatment can be simultaneously performed on the neck, the shoulders, the waist, the back and the buttocks only by one set of the prevention and treatment instrument.

Another Chinese patent 2,041,33662U on Portable console model cervical traction frame is issued to Chinese inventor. The patent discusses a kind of portable console model cervical traction frame, forms primarily of base, connecting rod, connecting rod and pulley adjusting rod. Base is T-Type structure, the middle part of T-shaped whippletree is porose, perpendicular the passing in hole of connecting rod and hole is fixed with bolt, described connecting rod is tubular structure, the other end of connecting rod has the nut of adjusting elasticity, the lower end of connecting rod is fixed in the upper end of connecting rod, and the near-end of pulley adjusting rod is plugged on the interior bolt of connecting rod and fixes, and the far-end of pulley adjusting rod is embedded with hauling block. Connecting rod due to this fraction frame is that two joints are adjustable, traction hight can be regulated arbitrarily as required, patient's horizontal position can loosen musculi colli completely, alleviate the spasm of musculi colli, remove the traction antagonism that traction seat muscular tone causes, horizontal position traction can slightly face upward stretch position under draw, be beneficial to the normal physiology of recovery cervical vertebra anteflexion, recover the mechanical balance of intra and extraspinal, the damage to soft tissue and intervertebral disc avoiding hyper-traction to cause, has good therapeutic effect.

Another device for treating neck-shoulder-pain bearing Chinese patent 2,274,044Y is issued to Chinese inventor. The utility model relates to a device for treating neck-shoulder-pain, which is characterized in that a pillow jaw belt is connected with a hang hammer by multiple-angle balance pulley block, a raising and lowering pulley block and a traction rope; the pillow jaw belt can be driven to wag up and down and left and right by the traction rope and the multiple-angle balance pulley block; a vertical frame chair is also provided with a propeller; an arm sleeve can be connected with the hang hammer by the traction rope and the pulley block. The utility model can effectively treat cervical spondylopathy and scapulohumeral periarthritis according to the disease symptom of patients; because the device for treating neck-shoulder-pain adopts the pillow jaw belt and the propeller, the device for treating neck-shoulder-pain can effectively prevent overtreatment, and can completely correct and change cervical vertebra deformation and the binding of shoulders.

Another Chinese patent 2,046,98780U on Automatic adjustable cervical traction device is issued to Chinese inventor. The patent discusses a kind of medical apparatus and instruments, particularly a kind of adjustable cervical traction device automatically, this traction apparatus is body structure, and automatically controlled part and mechanical drive department are placed in casing, and pulley is inlaid in the one end in guide post, traction electric machine is connected with controller, controller is connected with remote controller signal, and the guide post other end is provided with traction apparatus, and pull rope moves in guide post, the output shaft solderless wrapped connection of one end and traction electric machine, the other end is walked around pulley and is connected with traction apparatus; Traction electric machine is connected with actuator, and actuator is connected with controller; Regulating handle is positioned at casing outside and is connected with dancer rools, and dancer rools is toothed tooth bar, is placed in casing, and regulating handle is gear structure, regulates bottom and the regulating handle engaged transmission of rod, regulates the top of rod to be fixedly connected with guide post. This device manual adjustments combines with Electronic control and controls haulage, can control trailing weight and pull-in time simultaneously, and operation accurately, conveniently.

Another Chinese patent 1,053,61569A discussing a Combined neck-shoulder pillow relates to a combined neck-shoulder pillow, comprising a neck pillow portion, a head pillow portion and a shoulder pillow portion that can be assembled into a whole. The neck pillow portion is disposed on one or both of upper and lower sides of the head pillow portion, and the width of the shoulder pillow is equal to a sum of the widths of the head pillow portion and neck pillow portion; the height of the head pillow portion is less than that of the neck pillow portion and shoulder pillow portion. In this manner, the cervical vertebra can be kept in its normal physiological curvature while a user lies in the supine position; in addition, the shoulder pillow portion is disposed on the left and right sides of the head pillow portion of the combined neck-shoulder pillow, the shoulder pillow portion can provide forcible support for the head and neck of a user while the user lies on the side so as to keep the cervical vertebra kept in the normal position while the user is in the lateral position, and therefore the cervical vertebra is kept in the normal physiological status whether the supine position or the lateral position is used, the neck is given full rest and cervical health is benefited.

Another patent bearing U.S. Pat. No. 7,086,992B2 is issued to Jason Bowman Robert Gearhart Robert L. Richardson. The patent discusses a posture correction exercise device is disclosed to aid in correcting the common postural condition of kyphosis-lordosis by aiding in the exercise of the spinal erectors to strengthen the erectors to pull the user's spine and torso backward into normal alignment and by exercise of the mid-trapezious, rhomboid and posterior deltoid muscles to strengthen these muscles to pull the user's shoulder blades together and force the shoulders into normal alignment. The device operates by seating the user upon a declined seat to provide increased resistance by gravity. The device provides resistance to backward movement of user's body and resistance to backward rotation of user's arms. The hands of the user are positioned in supinated palms-up hand positions to increase the training effect of the backward rotation of the user's arms.

Another Head, neck and shoulder therapeutic exercise device bearing U.S. Pat. No. 5,116,359A is issued to MOORE JOSEPH L. The patent is about a head, neck and shoulder therapeutic exercise device which mechanically assists the human head and neck with intermittent rearward/forward movement in order to help correct or attenuate by means of muscular conditioning the cervical misalignment associated with forward head and flexible cervical lordosis. Mounted on a chair, an adjustable frame supports above the patient's head a motor assembly in fixed position and a motion-assist assembly which is horizontally moveable. The latter comprises in part a roller block with affixed roller bearing plate extending down behind the patient's head. The motor shaft, also behind the patient's head, has a cam which rides against a roller bearing in the roller bearing plate. As the cam's high side rotates against the bearing, the bearing plate moves away from the patient, thereby transferring rearward tension to the head by means of stretchable straps attached to said bearing plate and placed around the patient's forehead and chin. Each rotation of the cam is briefly halted by a timer when the head is in retracted position. After a holding phase, the rotation resumes, returning everything to original position. The head displacement distance is controlled by variously sized interchangeable cams. During therapy, the patient tries to do chin tuck exercises to accompany the cyclic mechanical movement.

Another Head and neck cushion bearing U.S. Pat. No. 4,494,261A is issued to WINSTON MORROW. The patent is about a composite head and neck cushion is provided for use by a person in a supine position. The cushion includes a first, resilient member having an upper surface which conforms to and supports the physiologic curvature of the cervical vertebrae. A second member supports the head in a raised, but unflexed position. The invention may alternatively be described as a method for cushioning the head and neck. The method comprises (1) resiliently supporting the back of the neck of the person in an elevated position while permitting the cervical vertebrae to maintain their normal, physiologic curvature, and (2) supporting the head of the person in an elevated position while maintaining the cervical vertebrae and the occiput in an unflexed, physiologic position.

A Cervical/occipital support bearing U.S. Pat. No. 4,708,129A is issued to Charles Pujals, Jr. The patent is about a one-piece contoured neck brace or support is disclosed which provides semi-rigid support for a user's head and neck by conforming to and contacting the occiput between the ears, the posterior and lateral neck area and the suprascapular region. The support may be retained on the user's neck by an adjustable strap or collar, and has forwardly extending portions to reduce any uncomfortable pressure on the user's throat. Ear protectors may be selectively attached to the support as needed, as may a head band. The head band may be used to further secure the support, the ear protectors, or both.

A Cervical pillow bearing U.S. Pat. No. 4,424,599A is issued to Nabil Hannouche. The patent is about a cervical pillow having a shoulder engaging portion, an occiput engaging portion, and a neck engaging portion therebetween. The cervical pillow provides continuous support from the shoulder portion to the occiputal portion of the user.

A Cervical/occipital support bearing U.S. Pat. No. 4,854,306A is issued to Charles Pujals, Jr. The patent is about a support is provided for a user's head and neck which provides varying degrees of support depending on the combination of support members utilized. A posterior shell which supports the occiput can be combined with either ear protectors or an anterior shell, or both and an overlying posterior shell can be utilized to provide increased support. A single posterior shell could include all support features such as the ear protectors formed as an integral port thereof. The anterior shell can have molded contours to increase its rigidity and jaw engaging side walls to reduce lateral movement.

A U.S. Pat. No. 4,793,334 also discusses neck support which incorporates a system and ties for tying down the structure to the body. A jawline bolster part is situated with respect to the structure to display development of the neck. The jawline bolster part is upheld on a swagger. The swagger is releasably and critically mounted to the system. A prop part stretching out from the swagger gives pivotable development of the swagger.

There are multiple solutions that have been presented in prior art. However, these solutions are limited and restricted to their conventional architecture, installation system and have considerable shortcomings which adversely affect the convenience with which they can be used. The prior systems have certain limitations including the design of assemblies which make them not suitable for every type of user. Moreover, the ease of use ability of these assemblies is also questionable.

It has to be noted that the current invention proposes an assembly with multiple advancements including a structure with symmetrical measurements in height and side width that make it more stable and easier to wear for the user. The proposed invention is a crafted from firm, polyurethane foam to give you the support you need for optimal tissue strengthening. The Occipital assembly with enlarged occipital nodes and increased firmness, is great for a variety of relaxation techniques, exercises and stretches that assist to restore and recover problematic soft tissues in the neck. This tool has a removable interlocking layer for height adjustability, providing a custom-tailored therapy session.

None of the previous inventions and patents, taken either singly or in combination, is seen to describe the instant invention as claimed. Hence, the inventor of the present invention proposes to resolve and surmount existent technical difficulties to eliminate the aforementioned shortcomings of prior art.

### SUMMARY

In light of the disadvantages of the prior art, the following summary is provided to facilitate an understanding of some of the innovative features unique to the present invention and is not intended to be a full description. A full appreciation of the various aspects of the invention can be gained by taking the entire specification, claims, drawings, and abstract as a whole.

The primary desirable object of the present invention is to provide a novel and improved form of a cervical orthotic assembly used to deal with Cervical Traction for Neck and Shoulder Pain.

It is another objective of the invention to provide an orthotic device which provides Chiropractic Alignment to support Trigger Point Therapy, Headaches, Migraine and Stress conditions.

It is further the objective of the invention to give an orthotic device which with its enlarged occipital nodes and increased firmness, provides variety of relaxation techniques, exercises and stretches that assist to restore and recover problematic soft tissues in the neck.

It is also the objective of the invention to provide a device of the type described which can be easily be adjusted to the fit accomplish the need.

It is further the objective of the invention to provide an enhanced Occipital Release Tool which counteracts the negative forces caused by forward head posture to help achieve proper cervical lordosis

It is also the objective of the invention to provide a Cervical Orthotic which provides a variety of relaxation techniques, exercises and stretches that assist to restore and recover a normal, healthy spinal curve.

It is further the objective of the invention to provide a firm, polyurethane foam to give support needed for optimal tissue strengthening.

It is moreover the objective of the invention to provide a removable interlocking layer for height adjustability, providing a custom-tailored therapy session as required.

It is another objective of the invention to provide a device that can be used for variety of factors but not limited to looking down at hand-held devices, sleeping with the head elevated excessively, or an acute injury such as whiplash.

It is moreover the objective of the invention to provide neck orthosis which is designed to be portable because of its light weight and can be taken to workspace, on long flights, road trips and anywhere else as required.

A further object is to provide an assembly that is simple, rapidly adjusted and easy to use.

It is also the objective of the invention to assist to use in seconds and effective for chronic neck pain, stress & tension headaches, poor sleep, shoulder tension and more.

It is moreover the objective of the invention which is great for athletes, bodybuilders, drivers, teachers, and anyone who needs a remarkable therapy session.

A still further object is to provide an assembly that is economical in cost to manufacture.

Other aspects, advantages and novel features of the present invention will become apparent from the detailed description of the invention when considered in conjunction with the accompanying drawings.

This Summary is provided merely for purposes of summarizing some example embodiments, so as to provide a basic understanding of some aspects of the subject matter described herein. Accordingly, it will be appreciated that the above-described features are merely examples and should not be construed to narrow the scope or spirit of the subject matter described herein in any way. Other features, aspects, and advantages of the subject matter described herein will become apparent from the following Detailed Description, Figures, and Claims.

### DETAILED DESCRIPTION

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

The present invention is directed to a occipital tool used to elicit Cervical Traction for Neck and Shoulder and Pain Chiropractic Alignment Pivot Device for Trigger Point Therapy Headaches Migraine and Stress.

Research finds, that the neck of people, shoulder, waist, back pain disease occurs mainly because of long time of sitting work, especially bend over one's desk working for a long time. Mechanical neck traction devices have been of immense value as an adjunct to the treatment protocols when treating neck-shoulder pain. On many occasions neck traction devices that are widely available on the market have been prescribed to patients to be used at home. All to date manufactured mechanical electrical or hydraulic neck traction devices require a person to either sit in a chair or lie down for the duration of the treatment. That is so because the traction force exerted onto the head comes from an electrical, mechanical or hydraulic source that is attached to either the traction bed or to the overhead console to which the traction source is fastened. That most times presents a problem and poor compliance of the patient; time is the most precious commodity we possess.

The current invention as per its preferred embodiments proposes a simple orthotic device which is engineered with curves, angles and ridges which is symmetrical in measurement. The firm polyurethane foam gives the support needed for optimal tissue strengthening. This tool has a removable interlocking layer for height adjustability, providing a custom tailored therapy session as per requirement.

The device as per its further embodiments allow three completely separate but attachable levels in curvilinear shape that allow the user to adapt each level to their specific need without having to but different sized blocks.

The Occipital Release Tool assembly as per its preferred embodiments is an assembly with enlarged occipital nodes and increased firmness which is great for a variety of relaxation techniques, exercises and stretches that assist to restore and recover problematic soft tissues in the neck.

The assembly further helps to restore issues associated with tension and stress which includes headache, cervical syndrome, tension at the base of the skull such as knotted muscles at the top of neck with just 10 minutes of use per day.

The assembly as per further embodiments allows to counter forward head posture. Typically, forward head posture can be attributed to daily habits such as looking down at a hand-held device, an improper sleep posture, or an acceleration/deceleration injury such as whiplash. The Occipital Release Tool counteracts the negative forces caused by forward head posture to help achieve proper cervical lordosis.

After receiving simple training and education, the patient will be able to properly position the device for low-stress, comfortable treatment at home.

The proposed assembly allows variety of relaxation techniques, exercises and stretches that assist to restore and recover a normal, healthy spinal curve. It further helps to restore issues associated with stiff neck, straight neck, military neck and forward head posture with just 10 minutes of use per day. It further releases neck and shoulder tension while soothing headaches and migraines.

The use of the device is simple the larger angled surface should face the patient's shoulders. For smaller patients or less aggressive treatment, user carefully removes sections by completing cut along the lines.

The device as per its further embodiments is crafted from firm, polyurethane foam to give the support needed for optimal tissue strengthening. The assembly has a removable interlocking layer for height adjustability, providing a custom-tailored therapy session as required. The neck orthosis is designed to be taken to the office, on long flights, road trips and just about anywhere. It is also small, lightweight, and easy to use which makes it the superior choice for on-the-go therapy sessions.

While a specific embodiment has been shown and described, many variations are possible. With time, additional features may be employed. The particular shape or configuration of the platform or the interior configuration may be changed to suit the system or equipment with which it is used.

Having described the invention in detail, those skilled in the art will appreciate that modifications may be made to the invention without departing from its spirit. Therefore, it is not intended that the scope of the invention be limited to the specific embodiment illustrated and described. Rather, it is intended that the scope of this invention be determined by the appended claims and their equivalents.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separately claimed subject matter.

I: An adjustable occipital tool apparatus comprising:
a body having symmetrical design;
body with occipital nodes at top;
removable height layers;
high density foam and
light weight and easy to carry

II: An adjustable occipital tool having two completely separate levels of care allowing the user to just remove the lower levels as needed to use the levels above.

III: An occipital tool of claim 1, wherein the surface area of the first support surface with occipital nodes comes in contact with the patient's neck and the base area is in contact with the substrate Surface. The tool has two completely separate levels of care that do not require cutting through each level to use it. The user just removes the lower layer as needed to use the main top layer.
